(19) United States
(12) Patent Application Publication
(10) Pub. No.: US 2004/0253185 A1
(43) Pub. Date: Dec. 16, 2004

(54) Medicated ink

(75) Inventors: Steve A. Herweck, Nashua, NH (US); Paul Martakos, Pelham, NH (US); Roger Labrecque, Londonderry, NH (US); Geoffrey Moodie, Nashua, NH (US)

Correspondence Address: LAHIVE & COCKFIELD, LLP., 28 STATE STREET, BOSTON, MA, 02109, US

(73) Assignee: ATRIUM MEDICAL CORP., Hudson, NH

(21) Appl. No.: 10/461,145

(22) Filed: Jun. 12, 2003

(51) Int. Cl. A61K 9/44; A61K 9/00

(52) U.S. Cl. 424/10.2

(57) ABSTRACT

A medical device is loaded with a number of therapeutic agents using a corresponding method to apply a medicated ink mark. The resulting medical device can include surface activation of an immobilizing medication, controlled medication release, and the ability to use dyes or pigments to delineate different active ingredients by location and dosage. The active medicinal compounds can be placed on selective areas of the medical device. The medical device having the medicated ink mark can provide a detectable and dosemetric controllable delivery to a specific targeted and localized location to provide the maximum therapeutic benefit. The medicated ink may be applied to the medical device by a number of different methods, by a manufacturer or by the user at the time of medical device use. Dimensions of the markings printed onto the medical device can further serve to control and identify to the user the dosage amount of the medical agent available on the marked medical device. Multiple types of medical agents with multiple application methods can be used. The medicated ink can be dyed, pigmented, or used as a colorless vehicle for the compound of interest, and can be formulated to incorporate either immobilized or exuding active agents onto the medical device.

### FIELD OF THE INVENTION

The present invention relates to a detectable drug exuding medicated ink that is applied to a medical device for therapeutic purposes.

### BACKGROUND OF THE INVENTION

Intravessel restenosis is the formation of neointimal tissue following a balloon or laser angioplasty and/or expandable stent placement within a hollow organ, such as a blood vessel. Within months of treating a coronary artery blockage with transluminal balloon angioplasty and/or deployment of a radially expandable tubular stent, platelet inflammation, platelet deposition, and cellular proliferation, migration, and matrix production occurs along the flow surface induced by the trauma of dilation and balloon expansion of an occluded vessel from a first smaller internal diameter to a second larger and radially distended diameter. Subsequently, the re-narrowing phenomena now known as “restenosis” occurs.

The occurrence of restenosis and smooth muscle cell proliferation following mechanical injury to endothelialized body fluid organ tissue can be significantly reduced, modulated, or eliminated, by use of localized drug delivery to the effected zone with immunosuppressive and chemotherapeutic drugs such as sirolimus, everolimus, tacrolimus, paclitaxel, or mycophenolic acid, which have all demonstrated anti-proliferative properties.

Medications to reduce restenosis have focused on administration of anti-platelet and anti-neoplastic agents, which either interfere with formation of thrombosis, platelet activation and deposition, or suppression of smooth muscle cell activation and/or proliferation, and localized cell death or apoptosis. Anti-coagulants commonly used for suppression of thrombosis include heparin, warfarin, low molecular weight heparin, hirudin (Lovqvist, A., et al., J. Int. Medicine, 233:215-116 (1993)) bavalirudin (Angiomix®). Agents for inhibiting the proliferation of smooth muscle cells include glucocorticoids, angiotensin converting enzyme inhibitors, colchicine, vincristine, actinomycin, low molecular weight heparin, platelet derived growth factor and others (Lovqvist, A., et al.). More recently, paclitaxel (Taxol®) and sirolimus (Rapamycin®) have been clinically proven effective at reducing, delaying and/or eliminating restenosis in coronary and peripheral vascular blood vessels (U.S. Pat. Nos. 5,616,608; 5,733,925; and 5,716,981).

One delivery method employed to deliver paclitaxel locally within a blood vessel to help control restenosis or smooth muscle cell hyperplasia, particularly with coronary arteries, is by use of a drug impregnated elastomeric polymer band. The band is bonded radially like a cigar band to the outer surface of a cylindrical, tubular, and mostly porous metal stent. Formed and bonded to a first smaller diameter of a porous metal tube, the elastomeric band stretches radially around the stent as the stent is expanded to a second enlarged fixed diameter inside a blood vessel, by inflation of a dilation or angioplasty balloon catheter. The plastically deformable metal struts in the wall of the stent permanently hold the stent in a fixed second larger diameter, which in turn, holds the drug impregnated radial elastomeric band in a second fixed diameter, while still remaining bonded and fixed to the outer surface of the porous metal stent. The drug impregnated polymer band allows the medication to leach, or elute, out from the polymer material and into the surrounding intraluminal contacting tissue after stent deployment and radial polymer band engagement with the tissue.

Drug impregnated elastomer polymer bands have been proven clinically to deliver medication to a localized area after stent deployment. However, such banding methods are not always practical due to the requirement of the radial elastomeric band to permanently bond to the stent and special requirements for stent placement without blocking all porous holes of the tubular stent. If the thickness of a stent with a fixed material band increases too much, the stent may become too thick for placement into a vessel lesion and/or not expand completely, rendering the stent undersized for the intended anatomical location. Such elastomeric polymer bands can cause significant flow turbulence along the inner surface of the stent device and block important side branches of the vessel following deployment, thus rendering portions of the porous metal stent non-porous. Therefore, increasing the mass or surface area of a drug immobilizing polymer material, or polymer band thickness and surface area coverage of the porous metal tube can have a dramatic effect on a stent's ability to be deployed or track along and fit into a narrow passageway of a stenotic tubular organ lesion. Further, such radial polymer banding methods can inhibit the ability of an expandable metal stent to expand uniformly from a small diameter to larger diameter.

Another method to help deliver medication for controlling restenosis or smooth muscle cell hyperplasia in the human coronary arteries entails the use of drug eluting coatings applied around the entire surface, or to one or more surfaces, of a tubular expandable stent device. With this method, the drug is impregnated or made part of the coating that is applied only to the surface of the porous metal tube-like paint.

A method for applying a drug to a stent is spray painting, or dipping, the stent into a bonding agent that contains a drug. These techniques can be made to coat preferred sections or a particular surface of the stent, or alternatively on all surfaces of the medical device. The coated or painted area is generally limited to the available surface area of the metal tubular surfaces.

Known coating methods provide drug release from a bonded polymeric material or coating that surrounds one or more surfaces of the stent that generally provide a fixed rate of release of one or more medications. Such techniques require immobilizing the active drug ingredient into the polymer coating bonding agent or polymer material prior to crimping and device fixation onto a delivery catheter. The drug containing coating, bonding agent, or polymer material is made part of the stent by fusing, impregnating, or bonding the medication containing polymer directly to the metal surface of stent, or in wells and/or holes provided in the metal stent wall, or by radial sleeve or elastomeric polymer attachment around the pores and struts of the metal tube stent, or by tubular and/or helical polymer sleeve methods whereby the drug eluting material surrounds a majority portion of the radial cylindrical surfaces of the stent in a spiral candy cane fashion.

In general, the methods that deliver a thicker coated, bonded, or sleeve material drug coating may limit the ability of the stent to uniformly expand to a desired fixed larger diameter due to increased wall thickness over the stent. The increased wall thickness and high surface profile can prevent a high profile compacted device from tracking properly, especially in tight lesions. Trackability, or the ability of the stent to pass along and through a narrow lesion, can be significantly reduced and hindered by the use of a thick, stiff, and high profile radial material, coating, and/or drug eluting polymer sleeve. Further, such non-bioerodible polymers tend to extend the foreign body reaction of the carrier polymer coating long after the medication has departed from the coating.

Typical polymer bonding, dip, or spray coatings experience a limited shelf life because such polymer drug coatings are applied to the stent prior to stent crimping, compaction, or fixation onto the delivery balloon catheter with a therapeutic half life of the drug or agent that is effected by the immobilizing polymeric coating. The amount of effective medication provided is often subject to the amount of medication that can be loaded into the polymeric coating material and the stability of the bonding agent after crimping, compaction, and fixation to the delivery catheter to avoid polymer cracking, delamination, or disruption. Often, such coatings experience microcracking of the drug-containing polymer following either crimping or expansion of a second larger fixed diameter. Medication stability after sterilization is another shelf-life limitation, as exposure to sterilization humidity and elevated temperatures often causes the immobilized drug to blush out of the polymer carrier to the surface of the bonding agent or coating, changing the intended release profile from the medical device.

Typical drug delivery coatings known in the art have no identification or detection means for the user of the medical device to distinguish one medication type from another or one dosage, class, or particular drug indication, from another. There is also no known dosage identification means provided on such drug eluting devices.

Currently known drug eluting medical devices, in particular stents, vascular grafts, rigid orthopedic and soft tissue implants do not provide physical evidence of a medication or identification means of the type and/or amount of medication applied to the medical device. Also, currently known drug eluting polymer application techniques for implantable devices, such as coronary stents, are applied to the porous metal tubes prior to crimping and/or compaction of the porous tubular stent onto, or into, a delivery catheter, balloon catheter, or guide wire. These medical delivery devices are required for mechanical deployment of a drug coated stent within the patient.

In addition, users of typical medical devices must rely solely on packaging material to identify type and quantity of medications found on any medical device for therapeutic treatment, dimensions, locations of the medicated areas applied to the medical device, or other pharmakinetic characteristics of a medication present with such devices. As such, the possibility of misuse or mislabeling exists, and the possibility of unknowingly switching devices previously removed from packaging during clinical use by the operator also exists. Users of some devices, such as a surgical mesh of PET, often must manually draw lines for guiding the cutting of a smaller swatch of mesh from a larger section to better fit a patient. Users of vascular grafts often cannot easily determine the outer diameter or other dimensions of a particular vascular graft that has been removed from its packaging. Users of a stent or catheter can also have difficulty in identifying the particular size of the device once the device packaging has been removed. If there is a drug or agent coating on the device, that too can be either undetectable, or difficult to detect, without some identification means.

### SUMMARY OF THE INVENTION

It is therefore desirable to mark or print a medicated ink with drug immobilizing and/or drug eluting properties onto a medical device with a means for the identification, detection, and confirmation of a medication on the medical device. This medicated ink technology provides a verifiable application means at the time of implant by the physician, eliminating the high cost of acquiring and maintaining expiring short shelf-life inventory problems currently incurred with those drug eluting coated stent devices known in the art. Conventional commercially available and research drug eluting coated stents have a maximum shelf life of only 6 months, making the costs for such therapeutic devices relatively expensive. The identification, detection, and confirmation of a medication applied to a medical device can be made visual to the human eye, or by other methods of detection. In addition, the present invention can provide a low cost and flexible means for marking and applying different amounts of a single medication, or for marking more than one medication at similar or different dosages, onto a medical device. The ability to mark a medication directly onto a medical device prior to use, during use, or after installation, further enhances the therapeutic performance of medical devices.

The present invention provides a medical device and methods to load the device with a variety of therapeutic agents. Surface activation of an immobilizing medication, controlled medication release, and the ability to use dyes or pigments to delineate different active ingredients, different locations, and different dosages on a device are all possible with the present invention. The invention also provides the ability to place, with specificity, the active medicinal compounds on selective areas of a medical device.

Medical devices used with a medicated ink mark can provide a detectable and dosemetrically controllable therapeutic agent or drug delivery means to a specific targeted and localized patient location to provide the patient with the maximum therapeutic benefit. The medicated ink can be applied to the medical device by a number of different methods, including but not limited to ink jet printer, marker pen, gas vapor deposition, roto gravure, spraying, painting, roller, blotting, dying, stamping, ink transferring, and ink pad or ink pad printing. The application of the medicated ink can be performed by the manufacturer, or by the user at the time of medical device use.

Dimensions of the markings printed onto the medical device can further serve to control and identify to the user the dosage amount of the medical agent available on the marked medical device. It should be appreciated that the present invention can be used with multiple types of medical agents and with multiple application methods, marking shapes, sizes, patterns, and orientations per medical device by the clinical user or by the medical device manufacturer. It should also be noted that the ink that is used can be dyed, pigmented, or used as a colorless vehicle for the compound of interest. The ink described in this invention can be formulated to incorporate immobilized and/or exuding active agents onto a medical device.

In accordance with one aspect of the present invention, a method of applying an identifiable and/or detectable medicated ink as a marking to an implantable medical device includes providing an applicator with the medicated ink. The applicator is used to apply a marking to the medical device to generating a specific dosage of a drug. Such visible and/or detectable marking can indicate a specific dosage of a drug, and type of medication. The dosage is controlled by a number of different visible and non-visual detection means, and/or detectable dimensions, of the medicated ink marking.

In accordance with one aspect of the present invention, a method of determining an amount of an identifiable and/or detectable medicated ink to be applied to a medical device includes determining the amount of medical agent to be applied to the device. The length and width of the medicated ink marking to be applied to the device is determined according to the amount of medical agent desired. A concentration and/or dilution of the medication and confirmed length and width of the medicated ink marking printed are applied to the device. Confirmation can be done visually, electronically, or by any means of identification or detection as understood by one of ordinary skill in the art.

In accordance with one embodiment, the present invention is designed for use with an implantable endoluminal stent structure, wherein the medicated ink contains a medical agent to limit restenosis or proliferation of tissue following vascular trauma by localized release of the medical agent when the stent is implanted within a body lumen, space, or cavity. Medical agents can be used with a number of different dry solid, gas transfer, deposition films, gel, or liquid medicated inks when printed onto the surface of a stent structure. The medical agents can include but are not limited to medications such as paxlitaxel, tacrolimus, everolimus, sirolimus, tissue plasmingen activators, nitric oxide donating derivatives, antibiotics, heparin, anti-thrombotics, anti-inflarnmatory agents, GP IIb/IIIa inhibitors, radiopaque or ultrasonic detectable dyes, and all cell permeation enhancing chemicals, enzymes, or agents.

In accordance with another embodiment of the present invention, a medical device includes a structure adapted for insertion into a patient. A detectable information conveying marking is applied to the structure. The marking contains a medical agent for contacting body fluid when the device is placed within a patient.

In accordance with various aspects of the present invention, the marking is applied with a marker. A dosage of the medical agent on the device can be determined by detection of the marking. A dosage of the medical agent on the device is controlled by detectable dimensions of the marking on the device. A dosage of the medical agent on the device can be determinable by visual detection of the marking. The device can include an additional marking where the original marking is in a first color and the additional marking is in a second color that differs from the first color. The marking can have more than one type of medical agent. The marking can be a therapeutic and/or a diagnostic medical agent. The medical device can be an implantable medical device, an indwelling medical device, a medical device having a therapeutic function, and/or a medical device having a diagnostic function. The medical device can be placed into a patient's body for permanent use, or for temporary use. The medical agent can include an antioxidant agent in the form of at least one of lazaroid, probucol, phenolic antioxidant, resveretrol, AGI-1067, and vitamin E; antihypertensive agents in the form of at least one of diltiazem, nifedipine, and verapamil; anti-inflammatory agents in the form of at least one of glucocorticoids, cyclosporine, and NSAIDS; growth factor antagonists in the form of at least one of angiopeptin, trapidil, and suramin; antiplatelet agents in the form of at least one of aspirin, dipyridamole, ticlopidine, clopidogrel, GP IIb/IIIa inhibitors, and abcximab; anticoagulant agents in the form of at least one of heparin, wafarin, hirudin, and bivalirudin; thrombolytic agents in the form of at least one of alteplase, reteplase, streptase, urokinase, and TPA; drugs to alter lipid metabolism in the form of at least one of fluvastatin, colestipol, atrovastatin, amlopidine, and lovastatin; ACE inhibitors in the form of at least one of elanapril, fosinopril, and cilazapril; antihypertensive agents in the form of at least one of prazosin and doxazosin; antiproliferatives and antineoplastics in the form of at least one of cochicine, mitomycin C, estradiol, everolimus, tacrolimus, paclitaxel, sirolimus, cilastozol, methatrexate, dexamethasone, doxorubicin, and mycophenolic acid; tissue growth stimulants in the form of at least one of bone morphogeneic protein and fibroblast growth factor; chemical donors of at least one of nitric oxide and super oxygenated O2; promotion of hollow organ occlusion or thrombosis agents in the form of at least one of alcohol, surgical sealant polymers, solyvinyl particles, 2-Octyl cyanoacrylate, hydrogels, and collagen; functional protein and factor delivery agents in the form of at least one of Insulin, Human Growth Hormone, estrogen, and nitric oxide; second messenger targeting agents in the form of at least protein kinase inhibitors; angiogenic agents in the form of at least one of angiopoetin and VEGF; anti-angiogenic agents in the form of at least endostatin; inhibition of protein synthesis agents in the form of at least halofuginone; antilnfective agents in the form of at least one of mupirocin, RIP, rifampin, ciprofloxacin, kanamycin, vancomycin, cefazolin, amikacin, cefiazidime, tobramycin, levofloxacin, silver, copper, hydryxapatite, penicillin, and gentamycin; gene delivery agents in the form of at least one of genes for nitric oxide synthase, human growth hormone, and antisense oligonucleotides; cell permeation enhanced medications, such as at least one of H2O, saline, and alcohol; nitric oxide donative derivatives in the form of at least NCX 4016; drug carrying nano-particles; drug carrying micro-spheres; liposomes, and/or imaging agents to identify and treat diseased areas, such as halogenated xanthenes, diatrizoate meglumine, diatrizoate sodium, and chemotherapeutic agents such as paclitaxel, cyclosporine, tacrilomus, fludarabine, doxorubicin, and sirolimus.

In accordance with further aspects of the present invention, the medical device can be in the form of a stent, a catheter, a vascular graft, a surgical mesh, and a medical device adapted for use external or internal to the patient. The medical agent can be suitable for release into the body of the patient, or release into tissue of the patient.

In accordance with another embodiment of the present invention, a method of applying a detectable medicated information conveying marking to an implantable medical device includes providing an applicator holding detectable medicated ink. A first marking of the detectable medicated ink is then applied to the implantable medical device to apply a specific dosage of a drug, wherein the dosage is controlled by the quantity of the first marking.

In accordance with further aspects of the present invention, the medical device can be pre-treated prior to applying the first marking. A second marking detectably different from the first marking can be applied to the medical device. Applying the first marking can include applying multiple drug medications to the implantable medical device. The markings can be applied by an ink jet printer, a marker pen, an ink pad device, by thermal transfer, a dry or moistened medicated ink wipe, and/or by gas vapor deposition.

In accordance with further aspects of the present invention, the first marking can be a first color, and a second marking can be formed of a second color that is visually or detectably differentiable from the first color.

In accordance with another embodiment of the present invention, a method of applying a medical agent to a medical device includes determining a length of detectable information conveying marking to be applied to the device according to the amount of medical agent desired. The determined length of marking is then applied to the device.

The device and/or targeted tissue treatment location can be further pre-treated for improved adhesion and therapeutic agent absorption prior to applying the medicated ink marking.

In accordance with further aspects of the present invention, a different marking that is detectably different from the marking can be applied. Applying the determined length of marking can include applying multiple drug medications to the medical device. The marking can be applied to the medical device by an ink jet printer, a marker pen, an ink pad device, using thermal transfer, and/or using gas vapor deposition.

In accordance with another embodiment of the present invention, a medicated stent system includes a stent structure adapted to be implanted in a patient. A detectable information conveying marking of ink is applied to the stent structure, wherein the ink contains a medical agent to limit tissue proliferation following vascular trauma and/or restenosis by release of the medical agent from the medicated ink when the stent is implanted. The therapeutic medication or drug agent can include at least one of paclitaxel, taxane, sirolimus, tacrolimus, everolimus, cilastozol, methatrexate, dexamethasome, estradiol, doxorubicin, cyclosporine, fluvastatin, lovastatin, atorvastatin, amlopidine, predinisone, phenolic antioxidant, reveratrol, AGI-1067, vitamin E, omega 3 fatty acids, RIP, and mycophenolic acid.

### BRIEF DESCRIPTION OF THE DRAWINGS

The invention will be more fully understood from the following detailed description taken in conjunction with the accompanying drawings, in which:

FIG. 1A is a perspective view of an example of application of medicated ink to a medical device in accordance with an exemplary embodiment of the present invention;

FIG. 1B is a cross-sectional view of the medical device of FIG. 1A;

FIG. 2A is a diagrammatic illustration of the medicated ink applied to a medical device in a circular pattern, in accordance with one aspect of the present invention;

FIG. 2B is a diagrammatic illustration of the medicated ink applied to a medical device in an annular pattern, in accordance with one aspect of the present invention;

FIG. 2C is a diagrammatic illustration of the medicated ink applied to a medical device in a spiral pattern, in accordance with one aspect of the present invention;

FIG. 2D is a diagrammatic illustration of the medicated ink applied to a medical device in a zigzag pattern, in accordance with one aspect of the present invention;

FIG. 2E is a diagrammatic illustration of the medicated ink applied to a medical device in letter form, in accordance with one aspect of the present invention;

FIG. 2F is a diagrammatic illustration of the medicated ink applied to a medical device in number form;

FIG. 2G is a diagrammatic illustration of the medicated ink applied to a medical device in a manner relaying dimensions of the device, in accordance with one aspect of the present invention;

FIG. 2H is a diagrammatic illustration of the medicated ink applied to a medical device in a manner providing an indication of how to implant the medical device 12 into a patient;

FIG. 21 is a diagrammatic illustration of the medicated ink applied to a medical device in a manner not readily discernable by the un-aided eye;

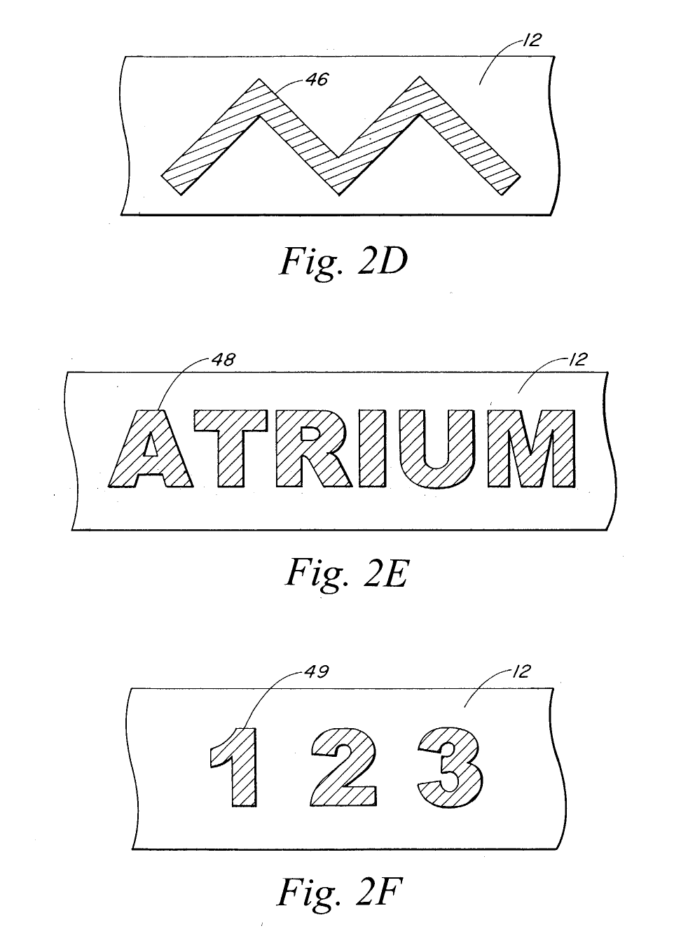

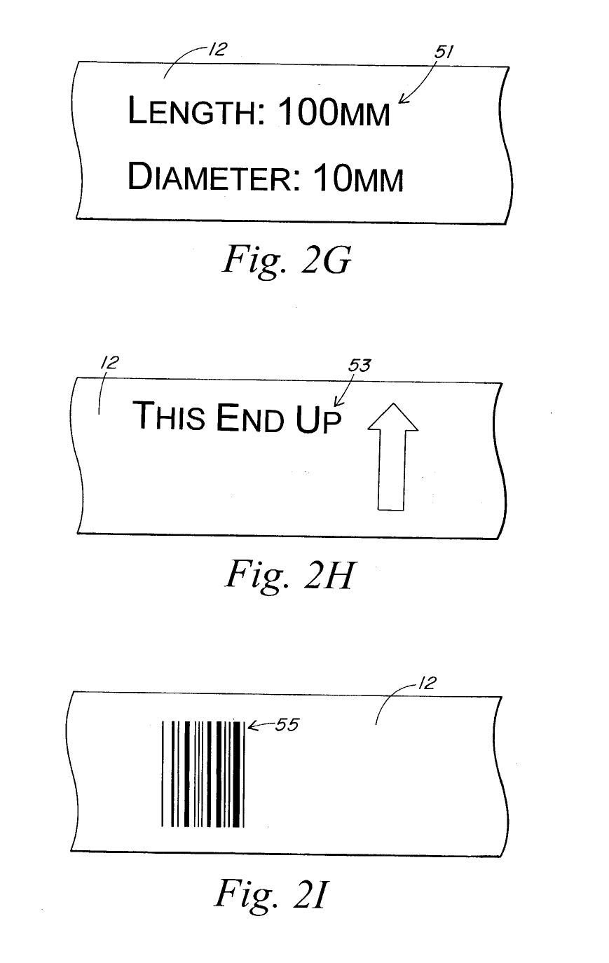

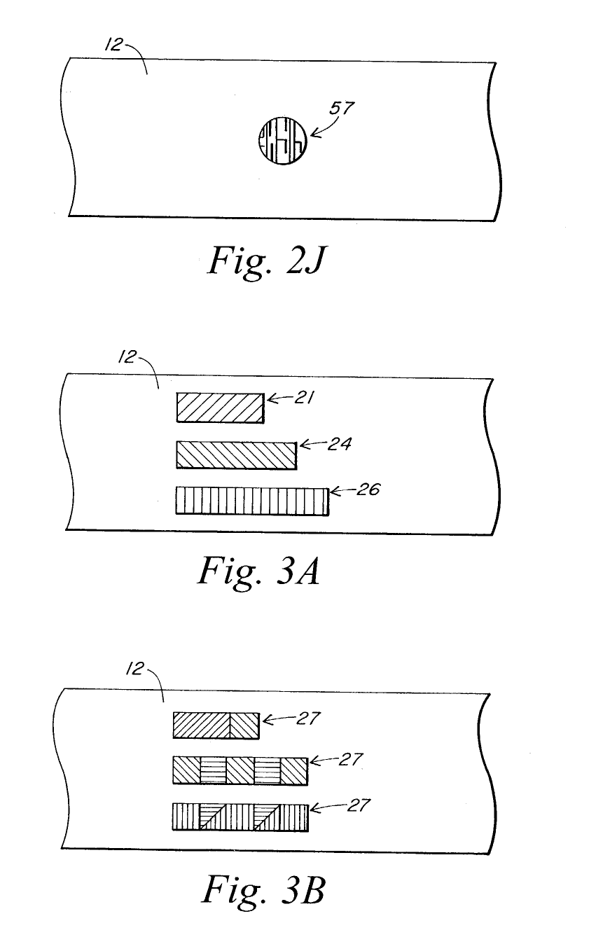

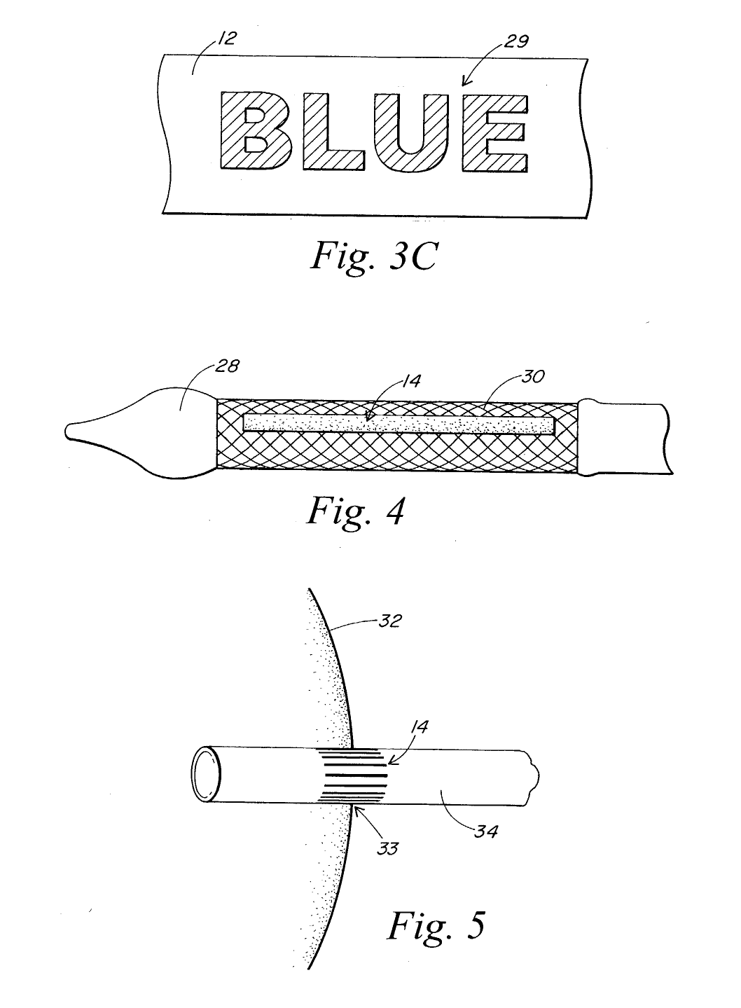

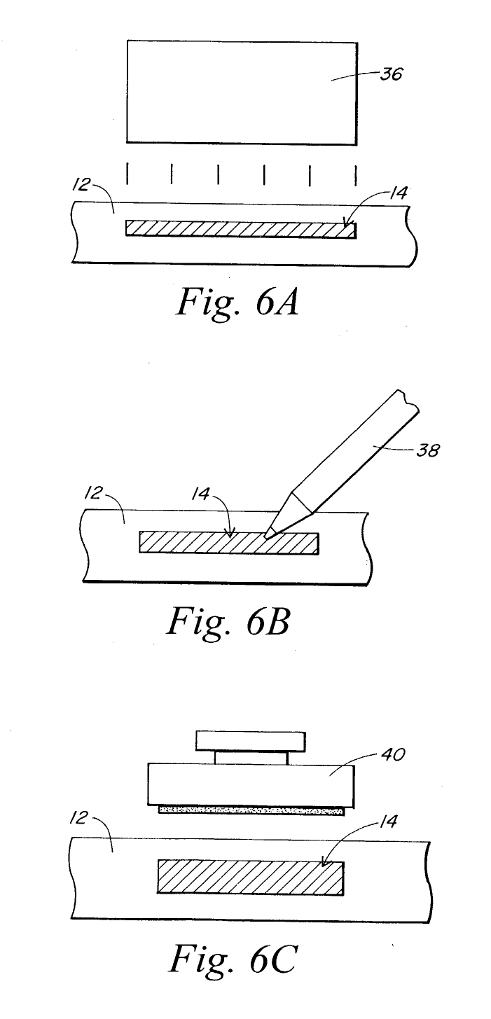

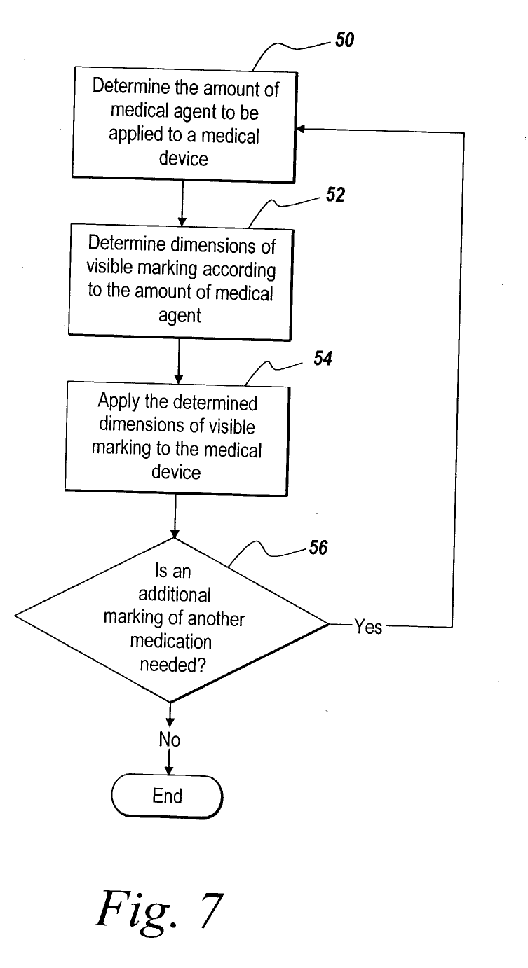

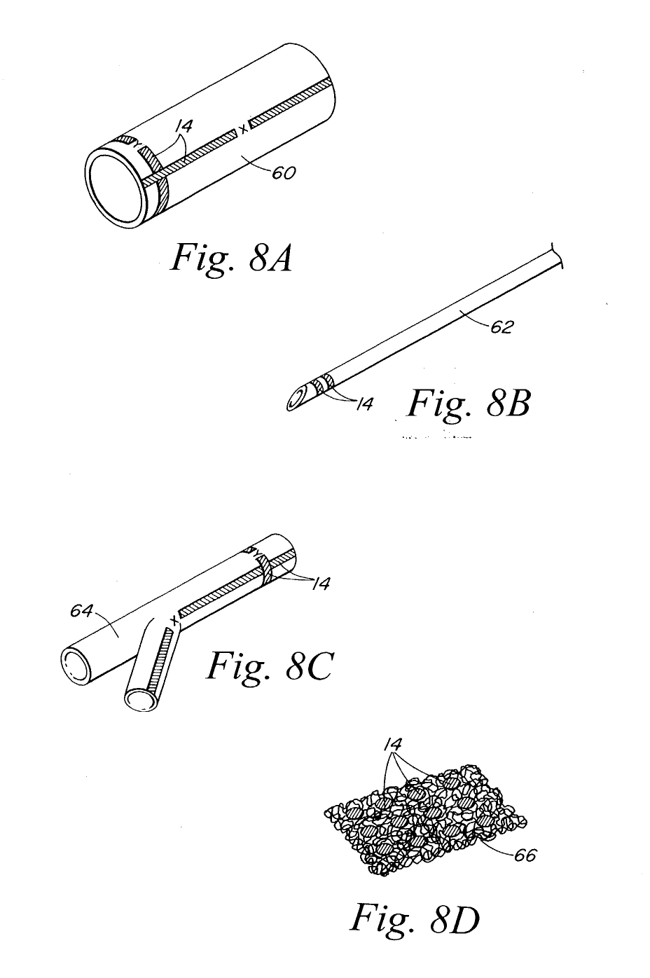

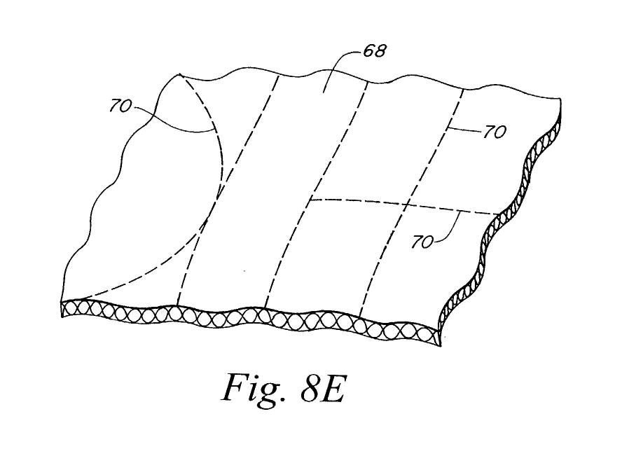

FIG. 2J is a diagrammatic illustration of the medicated ink applied to a medical device in a different manner not readily discernable by the un-aided eye;

FIG. 3A shows an example wherein multiple medical agents are applied to a single medical device, in accordance with one aspect of the present invention;

FIG. 3B shows another example wherein multiple medical agents are applied to a single medical device, in accordance with one aspect of the present invention;

FIG. 3C is a diagrammatic illustration of a medical device with color coded medicated ink markings in accordance with one aspect of the present invention;

FIG. 4 is a diagrammatic illustration of the medicated ink applied to a stent that is mounted on a balloon catheter;

FIG. 5 is a side view of a catheter where the medicated ink has been applied to the catheter;

FIG. 6A is a diagrammatic illustration of a first type of ink application device suitable for applying medicated ink, in accordance with one aspect of the present invention;

FIG. 6B is a diagrammatic illustration of a marker pen ink application device suitable for applying medicated ink, in accordance with one aspect of the present invention;

FIG. 6C is a diagrammatic illustration of an ink pad ink application device suitable for applying medicated ink, in accordance with one aspect of the present invention;

FIG. 7 is a flow chart illustrating the steps performed to determine an amount of a medical agent to be applied to a medical device, in accordance with one aspect of the present invention;

FIG. 8A is a diagrammatic illustration of a stent with medicated ink markings in accordance with one aspect of the present invention;

FIG. 8B is a diagrammatic illustration of a catheter with medicated ink markings in accordance with one aspect of the present invention;

FIG. 8C is a diagrammatic illustration of a vascular graft with medicated ink markings in accordance with one aspect of the present invention;

FIG. 8D is a diagrammatic illustration of a surgical mesh with medicated ink markings in accordance with one aspect of the present invention; and

FIG. 8E is a diagrammatic illustration of another surgical mesh with medicated ink markings in accordance with one aspect of the present invention.

### DETAILED DESCRIPTION

An illustrative embodiment of the present invention generally relates to improving the dosing and flexibility of adding different medications to an implantable or indwelling medical device. The present invention provides a clinical user with the opportunity to apply and confirm visually, electronically, or by other detection means, the type and/or dosage of medication applied to a medical device via a medicated ink. By use of a sterile medicated ink marker, the user can actually apply and control the amount of drug or dose of drug marked on to the implantable medical device prior to insertion or medical device installation. Alternatively, the markings can be placed on the device by a manufacturer. Detectable marked dimensions and/or color of a medicated ink marking on the medical device serves to identify and help control the prescribed dosage amount of the medical agent when applied to the medical device by the manufacturer and/or clinical user. The markings can relay a variety of information, such as dimensions, drug information, other medical device characteristics, pattern guidelines, and other usage instructions, if desired.

The information conveying medicated markings are identifiable or detectable. What is meant by identifiable and detectable is that the medicated markings are not necessarily visible to the un-aided eye, and the information stored within the markings is not necessarily discernable with the un-aided eye. More specifically, the information conveying markings can be visually based, such as with specific colors, symbols, patterns, and the like.

Alternatively, the information conveying markings can be invisible or substantially invisible to the un-aided eye, but can be made visible using any number of devices. For example, the markings can utilize ink that can only be seen if doused in a developing type solution that chemically alters the appearance. The markings can utilize ink that is only visible when, e.g., an infra read or ultra violet, or some other specific wavelength of light is shining on the ink. The markings can also be made visible when a specific temperature of the ink is achieved.

In addition, the information conveying markings can be visible, but not readily discernable. For example, the markings can take the form of a bar code, or some other machine vision based code. Such markings are visible, but without electronic or digital translation, the information conveyed by the marking is not readily discernable.

All of the above instances are intended to fall under the general scope of the terms identifiable and detectable as utilized herein. In addition, the markings convey various forms of information useful to the user, as detailed herein below.

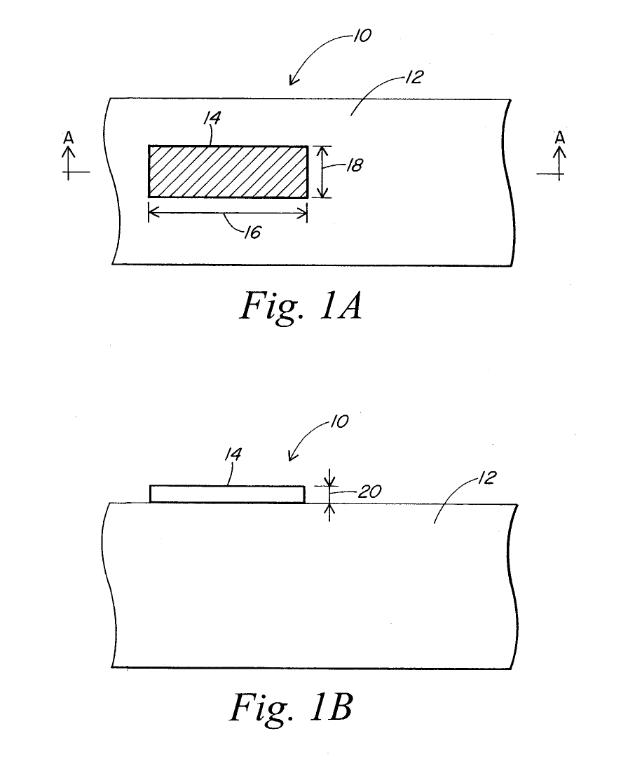

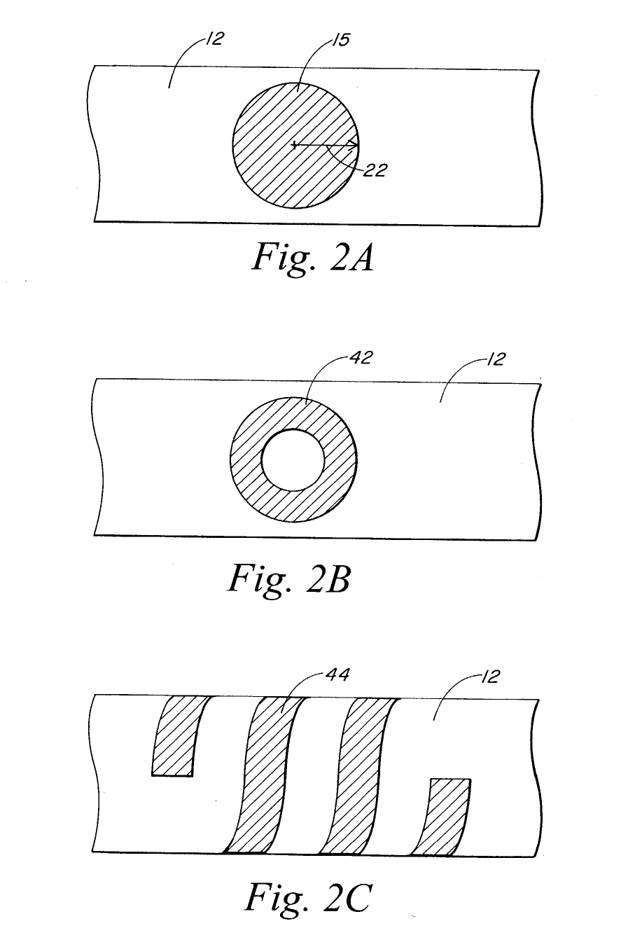

FIGS. 1A through 8E, wherein like parts are designated by like reference numerals throughout, illustrate example embodiments of a medicated ink based drug delivery system according to the present invention. Although the present invention will be described with reference to the example embodiments illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

The teachings of the present invention are applicable both to temporary and permanent use medical devices. A temporarily-placed medical device is defined as being a device that can be removed or degrades at the conclusion of the therapeutic or diagnostic purpose. A permanently-placed medical device, in contrast, stays within the body for an extended period of time, or in perpetuity.

The exemplary embodiments of the present invention provide a controllable and dosemetric means for identifying a medication, and/or identification of its dose or release rate at a specific area where the ink mark denotes the drug exuding location on the medical device. Examples of a medical device that can be used with the present invention include but are not limited to a stent, a staple, a suture, a needle, a catheter, a microsphere, a bulking agent, a valve, a pacemaker, and electronic sensor, an electrode, a port, a soft tissue implant, a bony tissue implant, a bone growth stimulating implants, a vessel puncture closure device, a vascular graft, a surgical fabric, a surgical mesh, a bladder suspension device, a tissue augmentation device, a hernia plug, a breast implant, other prosthetic implants, and any medical device that remains in contact with body tissue or body fluids sufficiently adequate to impart activation of and/or release of the medication into the localized body tissue or body fluid from the medicated ink.

FIGS. 1A and 1B illustrate examples wherein a medicated ink is applied to a medical device. FIGS. 1A and 1B show a medicated ink marking 14 that has been applied to a medical device 12. The medicated ink marking 14 is made by applying a medicated ink that includes an ink carrier component, a medical agent component, and optionally an adhesive or bonding agent for extended or permanent ink adhesion to the medical device. Medication saturation, loading, and dimensions of the medicated ink marking 14 control the dosage of the drug that is delivered to the patient. The ink can be made visible, or alternately detectable, by accessory device means that applies the ink so that the user can confirm the application and the appropriate dosage applied to the medical device. The ink may be visible either to the naked eye, under illumination by selected types of light, or when the user employs accessory detection aids (such as electronic scanner). The dosage of available medication can also be visibly identified by color or by combination with the dimensions and/or light refraction of the medicated ink marking 14.

The medicated ink marking 14 can be applied to the medical device 12 in various shapes and forms. FIGS. 1A and 1B show an example where the medicated ink is applied to the medical device 12 in the form of the medicated ink marking 14. The medicated ink marking 14 results from applying a medicated ink that includes an ink component and a medical agent component. In one embodiment, the amount of medical agent in the medicated ink marking 14 corresponds to the dimensional volume of the ink marking. The dimensional volume of ink applied in FIGS. 1A and 1B is equal to the product of length 16, width 18, and height 20 of the marking. The amount of medical agent on the medical device 12 may thus be controlled by varying the dimensions of the medicated ink marking 14. For example, the amount of medicated ink on the medical device may be varied by varying the length 16 of the medicated ink marking 14, the width 18 of the medicated ink marking, or the height 20 (i.e., thickness) of the medicated ink marking. The medicated ink marking can further be printed in a geometric code or universal bar code format for identification and detection of the medication applied onto a medical device. The amount of medicated ink deposition onto a medical device can further be increased by altering the surface chemically or topographically with wells, surface depressions, raised ridges and valleys, or with microscopic or nano-size pores.

The surface area of the medicated ink marking 14 can also affect the rate of delivery of the medical agent to the patient. In general, a larger surface area results in a higher rate of delivery of the medical agent than a smaller surface area (given a same concentration of medical agent). Further, an irregular surface topography including wells, holes, valleys, ridges, or microscopic or nano-size pores may be used to either increase or decrease the amount of medicated ink applied to the medical device. Hence, a physician or manufacturer may wish to consider both the volume and surface area when marking a medical device with a medicated ink.

Combined use of non-medicated ink to form blended ink with the medicated ink is another method to control the rate of delivery of the medical agent to the patient. With the addition of the non-medicated ink, the amount and rate of activation and/or release of the medical agent can be made different for different medical devices, different medical agents, different anatomical locations, and/or different device applications. A second non-medicated ink can further be applied as a second marking step to modulate the activation and/or release of the medical agent from the medicated ink. In addition, the medical device can be pre-treated with a medicated or non-medicated substance.

Those skilled in the art will appreciate that a number of different bio-erodable, soluble, or permanent marker inks may be used to create the medicated ink marking 14. In general, inks are formulated using a pigment to impart color, a resin binder to form the finished ink and carry the pigment, drug exuding medication, or chemical and/or solvent required to enable the binder-pigment mixture to be adhered to the medical device printed. Suitable pigments include but are not limited to those approved by the USFDA for medical use as listed in Title 21, Sections 73 and 74 of the Code of Federal Regulations (CFR). The following are directly applicable to medical devices:

| | |
|---|---|
| Ultramarine blue | FD&C Blue |
| Iron oxide | FD&C Green |
| Titanium oxide | FD&C Red |
| Chromium-cobalt-aluminum oxide | FD&C Yellow |
| Ferric ammonium citrate | D&C Orange |
| Chromium oxide green | D&C Brown |
| Logwood extract | D&C Violet |
| Phthalocyanine green | |

Medical agents may be added directly to ink formulations to form medicated ink. Additives and drug carrying nano-particles or microspheres containing medical agents may also be included in the medicated ink formulation to achieve specific rates of medication permeation to local tissue. For example, fast soluble and slow soluble nano-particles or microspheres, organic solvents, and surfactants may be used to achieve a desired medicated ink viscosity to apply the ink onto a substrate. The solvent and surfactant are optionally removed in a subsequent process step. Other additives can include plasticizers, bio-erodable components, dye components, adhesives, bonding agents, medication stabilizers, coated and non-coated medical agent nano-particles, or microspheres, designed to improve the ink's flexibility, flow, pigment stability, shelf-life stability, and rate of surface activation and/or release into tissue or body fluid. Medicated inks can also be formulated containing liposomes, with medication enclosed in liposomes, or phospholipid coatings. These inks can be triggered to release active compounds using an internal or external stimulus, such as ultrasound.

The following examples illustrate exemplary embodiments of the present invention.

### EXAMPLE #1

A medicated ink was formulated using chromium-cobalt-aluminum oxide pigment (cobalt blue-CFR 73.1025); ethyldiglycolacetate (CAS#112-15-2) and aromaic hydrocarbons (CAS#64742-95-6) solvent; cellulose and kaolin (CAS#1332-58-7) binders in a liquid base consisting of ethylene glycol monoethyl ether acetate (CAS#111-15-9), butyl acetate (CAS#123-86-4) and aromatic petroleum distillates (CAS#64742-95-6); Rapamycin (China Chemical Synthesis lot #89116003).

The solution was blended to achieve a homogenous mixture and used to print a pattern on a coronary stent platform (i.e., the Atrium Medical Flyer stent). For this example, the amount of Rapamycin contained in the print pattern on the stent was calculated to be 0.041 mg (˜41 ug).

Bare (non-medicated ink stents) and printed (stents containing medicated ink) were evaluated for effect on smooth muscle proliferation in cell culture. The following graph (Graph #1) shows that stents that were marked with the medicated ink significantly reduced smooth muscle cell proliferation compared to non-medicated non-marked stent controls.

[image]

### EXAMPLE #2

A medicated ink was formulated using chromium-cobalt-aluminum oxide pigment (cobalt blue-CFR 73.1025); ethyldiglycolacetate (CAS#112-15-2) and aromaic hydrocarbons (CAS#64742-95-6) solvent; cellulose and kaolin (CAS#1332-58-7) binders in a liquid base consisting of ethylene glycol monoethyl ether acetate (CAS#111-15-9), butyl acetate (CAS#123-86-4) and aromatic petroleum distillates (CAS#64742-95-6); Rapamycin (China Chemical Synthesis lot #89116003). Rectangular ePTFE pledgets (0.40″×0.25″) were pad printed with the medicated ink and allowed to dry. The ink coating weight was calculated and the samples were put into a dissolution test using 1.8 ml of Nerl water. The samples were tested for Rapamycin release at periodic intervals using HPLC, with the results being shown in Graph #2.

[image]

### EXAMPLE #3

A transparent medicated ink was formulated using Poly (DL-Lactide-co-Caprolactone), Methylene Chloride, ethyldiglycolacetate (CAS#112-15-2) and aromaic hydrocarbons (CAS#64742-95-6) solvent and Rapamycin (China Chemical Synthesis lot #89116003). Rectangular ePTFE pledgets (0.40″×0.25″) were pad printed with the medicated ink and allowed to dry. The ink coating weight was calculated and the samples were put into a dissolution test using 1.8 ml of Nerl water. The samples were tested for Rapamycin release at periodic intervals using HPLC, with the results as shown in Graph #3.

[image]

### EXAMPLE #4

Rapamycin (China Chemical Synthesis lot #89116003) was dissolved in ethanol at a concentration of 10 mg/ml. The tip of a marker pen was then soaked over night in the drug solution and then placed back in the marker pen. The marker pen was used to mark rectangular ePTFE pledgets (0.40″×0.25″) which were then put into dissolution. Rapamycin concentration was determined using HPLC. After one day, the samples had released an average of 2.1 micrograms of rapamycin. After three days, the samples had released an average total of 2.5 micrograms of rapamycin.

### EXAMPLE #5

A medicated ink was formulated using chromium-cobalt-aluminum oxide pigment (cobalt blue-CFR 73.1025); ethyldiglycolacetate (CAS#112-15-2) and aromaic hydrocarbons (CAS#64742-95-6) solvent; cellulose and kaolin (CAS#1332-58-7) binders in a liquid base consisting of ethylene glycol monoethyl ether acetate (CAS#111-15-9), butyl acetate (CAS#123-86-4) and aromatic petroleum distillates (CAS#64742-95-6); rapamycin (China Chemical Synthesis lot #89116003). The tip of a marker pen was then soaked over night in the drug solution. The tip was then placed back in the marker pen. The marker pen was used to mark rectangular ePTFE pledgets (0.40″×0.25″) which were then put into dissolution. Rapamycin concentration was determined using HPLC. After one day in dissolution, the sample had released an average of 6.6% of the total calculated Rapamycin. After three days in dissolution the samples had released an average total of 9.6% of the total calculated Rapamycin.

### EXAMPLE #6

A transparent medicated ink was formulated using Poly (DL-Lactide-co-Caprolactone), Methylene Chloride, ethyldiglycolacetate (CAS#112-15-2) and aromaic hydrocarbons (CAS#64742-95-6) solvent and Rapamycin (China Chemical Synthesis lot #89116003). The tip of a marker pen was then soaked over night in the drug solution. The tip was then placed back on the marker pen. The marker pen was used to mark rectangular ePTFE pledgets (0.40″×0.25″) which were then put into dissolution. Rapamycin concentration was determined using HPLC. After one day in dissolution, the sample had released an average of 11.1% of the total calculated Rapaamycin. After three days it had released an average total of 13.6% and after 6 days in dissolution it had released an average total of 14.7% of the total calculated Rapamycin.

Those skilled in the art will appreciate that a number of different medical agents may be used in the medicated ink marking 14. For example, anesthetic, anti-infective, lipid lowering, absorption enhancing, anti-oxidant, anti-platelet, cytostatic or cytotoxic medications can be used. In addition, medical agents that promote hollow fluid organ vaso dilation, vaso constriction, occlusion, or thrombosis can be used. The medical agents may include drugs that promote anti-thrombotic activity or can be a clot lysing agent known as a thrombolytic. The medical agents can be kinases or enzymes. The medical agents can be those that promote anti-inflammatory activity or those that promote or stimulate new bone growth. The medical agents can further include agents that promote new cell growth and/or tissue regeneration. The table below (Table #1) summarizes some examples of suitable therapeutic medication agents listed by drug class.

TABLE #1

| CLASS | EXAMPLES |
|---|---|
| Antioxidants | Alpha-tocopherol, lazaroid, probucol, phenolic antioxidant, |
| | resveretrol, AGI-1067, vitamin E |
| Antihypertensive Agents | Diltiazem, nifedipine, verapamil |
| Antiinflammatory Agents | Glucocorticoids, NSAIDS, ibuprofen, acetaminophen, |
| | hydrocortizone acetate, hydrocortizone sodium phosphate |
| Growth Factor | Angiopeptin, trapidil, suramin |
| Antagonists | |
| Antiplatelet Agents | Aspirin, dipyridamole, ticlopidine, clopidogrel, GP IIb/IIIa |
| | inhibitors, abcximab |
| Anticoagulant Agents | Bivalirudin, heparin (low molecular weight and |
| | unfractionated), wafarin, hirudin, enoxaparin, citrate |
| Thrombolytic Agents | Alteplase, reteplase, streptase, urokinase, TPA, citrate |
| Drugs to Alter Lipid | Fluvastatin, colestipol, lovastatin, atorvastatin, amlopidine |
| Metabolism (e.g. statins) | |
| ACE Inhibitors | Elanapril, fosinopril, cilazapril |
| Antihypertensive Agents | Prazosin, doxazosin |
| Antiproliferatives and | Cyclosporine, cochicine, mitomycin C, sirolimus |
| Antineoplastics | microphenonol acid, rapamycin, everolimus, tacrolimus, |
| | paclitaxel, estradiol, dexamethasone, methatrexate, |
| | cilastozol, prednisone, cyclosporine, doxorubicin, |
| | ranpirnas, troglitzon, valsarten, pemirolast |
| Tissue growth stimulants | Bone morphogeneic protein, fibroblast growth factor |
| Gasses | Nitric oxide, super oxygenated O2 |
| Promotion of hollow | Alcohol, surgical sealant polymers, polyvinyl particles, 2- |
| organ occlusion or | octyl cyanoacrylate, hydrogels, collagen, liposomes |
| thrombosis | |
| Functional Protein/Factor | Insulin, human growth hormone, estrogen, nitric oxide |
| delivery | |
| Second messenger | Protein kinase inhibitors |
| targeting | |
| Angiogenic | Angiopoetin, VEGF |
| Anti-Angiogenic | Endostatin |
| Inhibitation of Protein | Halofuginone |
| Synthesis | |
| Antiinfective Agents | Penicillin, gentamycin, adriamycin, cefazolin, amikacin, |
| | ceftazidime, tobramycin, levofloxacin, silver, copper, |
| | hydroxyapatite, vancomycin, ciprofloxacin, rifampin, |
| | mupirocin, RIP, kanamycin, brominated furonone, algae |
| | byproducts, bacitracin, oxacillin, nafcillin, floxacillin, |
| | clindamycin, cephradin, neomycin, methicillin, |
| | oxytetracycline hydrochloride. |
| Gene Delivery | Genes for nitric oxide synthase, human growth hormone, |
| | antisense oligonucleotides |
| Local Tissue perfusion | Alcohol, H2O, saline, fish oils, vegetable oils, liposomes |
| Nitric oxide Donative | NCX 4016 - nitric oxide donative derivative of aspirin, |
| Derivatives | SNAP |
| Gases | Nitric oxide, super oxygenated O₂ compound solutions |
| Imaging Agents | Halogenated xanthenes, diatrizoate meglumine, diatrizoate |
| | sodium |
| Anesthetic Agents | Lidocaine, benzocaine |
| Descaling Agents | Nitric acid; acetic acid, hypochlorite |
| Chemotherapeutic Agents | Cyclosporine, doxorubicin, paclitaxel, tacrolimus, |
| | sirolimus, fludarabine, ranpirnase |
| Tissue Absorption | Fish oil, squid oil, omega 3 fatty acids, vegetable oils, |
| Enhancers | lipophilic and hydrophilic solutions suitable for enhancing |
| | medication tissue absorption, distribution and permeation |
| Anti-Adhesion Agents | Hyalonic acid, human plasma derived surgical |
| | sealants, and agents comprised of hyaluronate and |
| | carboxymethylcellulose that are combined with |
| | dimethylaminopropyl, ehtylcarbodimide, hydrochloride, |
| | PLA, PLGA |
| Ribonucleases | Ranpirnase |
| Germicides | Betadine, iodine, sliver nitrate, furan derivatives, |
| | nitrofurazone, benzalkonium chloride, benzoic acid, |
| | salicylic acid, hypochlorites, peroxides, thiosulfates, |
| | salicylanilide |

In addition to or in conjunction with the above table, the medical agent of the present invention can further include an antimicrobial agent. As utilized herein, the term antimicrobial agent shall include antibiotic, antimicrobial, antibacterial, germicidal agents and the like. There may be a combination of antimicrobial agents. In addition, example antibiotics which may be used in conjunction with the present invention include: aminoglycosides, such as gentamicin, kanamycin, neomycin, paromomycin, streptomycin, or tobramycin; ansamycins, such as rifamycin, or rifampin; cephalosporins, such as cephalexin, cephaloridine, cephalothin, cefazolin, cephapirin, cephradine, or cephaloglycin; chloramphenicols; macrolides, such as erythromycin, tylosin, oleandomycin, or spiramycin; penicillins, such as penicillin G and V, phenethicillin, methicillin, oxacillin, cloxacillin, dicloxacillin, floxacillin, nafcillin, ampicillin, amoxicillin, or carbenicillin; suflonamides; tetracyclines, such as tetracycline, oxytetracycline, chlortetracycline, methacycline, demeclocycline, rolitetracycline, doxycycline, or minocycline; trimethoprim-sulfamethoxazole; polypeptides, such as bacitracin, polymyxins, tyrothricin, or vancomycin; and miscellaneous antibiotics, such as lincomycin, clindamycin, or spectinomycin, in addition to oxytetracycline hydrochloride (OTC).

There are a plurality of germicides which may at least partially form the medical agent of the present invention, including phenols; cresols; resorcinols; substituted phenols; aldehydes; benzoic acid; salicyclic acid; iodine; iodophors, such as betadine; chlorophors, such as hypochlorites; peroxides; such as hydrogen peroxide and zinc peroxide; heavy metals and their salts, such as merbromin, silver nitrate, zinc sulfate; surface-active agents, such as benzalkonium chloride; furan derivatives, such as nitrofurazone; sulfur and thiosulfates; salicylanilides; and carbanilides.

The amount of the antibiotic or germicide present in an application of a marking varies with the nature of antibiotics or germicides employed and to some extent the method applying the marking as understood by one of ordinary skill in the art.

As mentioned above, the medicated ink marking 14 may have a number of different detectable or visible shapes, FIG. 2A illustrates an example of the medicated ink marking 14 in the form of a circular shape medicated ink mark 15 of radius 22 applied to the medical device 12. FIG. 2B illustrates another example of the medicated ink marking 14 in the form of an annular shaped medicated ink marking 42. FIG. 2C shows an additional example where the medicated ink marking 14 is in the form of a helical spiral stripe medicated ink marking 44 that extends around the circumference and the length of the medical device 12. FIG. 2D shows an example where the medicated ink marking is applied as a zigzag medicated ink marking 46 on the medical device 12. This form of medicated ink marking 14 can signify a brand name of the drug or agent. Likewise, a corresponding brand symbol or trademark can also be included to portray a brand identity. FIG. 2E shows another example where the medicated ink marking 14 is applied in letter form to create an alpha medicated ink marking 48 on the medical device 12. FIG. 2F shows another example where the medicated ink marking 14 is applied in number form to create a numeric medicated ink marking 49 on the medical device 12. FIG. 2G shows an example embodiment where the medicated ink is applied in an alphanumeric format to create the medicated ink marking 14 in the form of an alphanumeric medicated ink marking 51, conveying dimension information about the medical device 12. FIG. 2H shows an example embodiment where a medicated ink mark 53 provides an indication of how to implant the medical device 12 into a patient.

FIGS. 2I and 2J show additional example embodiments where the medicated ink is visible, with and/or without an accessory device, but is not readily discernable as information to the user. More specifically, FIG. 2I shows a medicated ink mark 55 that forms a bar code readable by an infrared scanner. FIG. 2J shows a medicated ink mark 57 that forms a machine vision code readable by use of machine vision devices, as understood by one of ordinary skill in the art.

Numerous modifications to medicated ink marking shape, including pattern and orientation, will be apparent to those skilled in the art in view of the foregoing description. Accordingly, this description is to be construed merely as illustrative of the inventive concept herein. The description and illustrations should not be construed as limiting the invention.

FIG. 3A illustrates an example where multiple types of medical agents are applied to a single medical device. Use of different drugs can be further distinguished by use of different detectable methods or visible colors for different classification types of medications. FIG. 3A shows the medical device 12 having the medicated ink marking 14 in the form of a blue medicated ink mark 21 for immunosuppressive drugs, a red medicated ink mark 24 for anticoagulants, and a yellow medicated ink mark 26 for cytostatic medication. The use of different colors allows a physician, or other clinical user, to visibly identify the class of medication applied to a medical device prior to implantation or device use. The different color schemes for different classification types of medication provide the user with the ability to check and confirm prior to installation which medication or therapeutic application is incorporated into the ink applied to the medical device. Further, the medicated ink markings 14 have different dimensional lengths that are chosen for specific dosages for each corresponding medical agent. The specific color scheme utilized can be standardized by, for example, a national standardizing entity. The color scheme can include solid colors, as shown in FIG. 3A, or can include simple patterns of alternating or otherwise differing colors, as shown in markings 27 of FIG. 3B. One of ordinary skill will appreciate the virtually infinite variability of colors, hue, fluorescence, and simple color patterns that can be used to identify particular classes or types of drugs. The colors can identify specific brand names of drugs, or any other desired clinically related attribute, as well.

FIG. 3C depicts a further example embodiment of the present invention. The medicated ink marking 14 is embodied as a color-coded medicated ink mark 29 in the color of blue. Other colors can be embodied in a similar manner in accordance with the teachings of the present invention as understood by one of ordinary skill in the art.

Further, the medicated ink markings 14 all have different lengths and thicknesses chosen for delivery of the appropriate dosages of the medical agents. In other words, given a uniform number of application layers, increased lengths of medicated ink markings 21, 24, and 26 result in increased dosages of the medical agents. Therefore, upon quick visual inspection, a user can determine the dosage amount provided on a particular medical device, without having to refer back to previously removed packaging. If the thickness is varied, the same length of marking 21, 24, and 26 can also result in different dosages.

As mentioned above, the medicated ink markings 14 may be applied to a number of implantable and indwelling types of medical devices. FIG. 4 illustrates a crimped stent 30 on a balloon catheter 28 with the medicated ink marking 14 applied thereon. Marking the surface of a medical device with identifiable and/or detectable medicated ink does not affect the uniform expansion or plastic deformation of a porous metal cylinder stent structure 30. The present invention does not sacrifice a stent's flexibility and trackability when the identifiable and/or detectable medicated ink mark is made on the outer surface of the stent structure 30. The present invention also does not limit the stent's ability to uniformly expend to a desired fixed larger diameter. In addition, any type of stent can be medicated just prior to use, substantially lowering the treatment cost to the patient, and the cost of the final product, and further extending the shelf life of the medical devices or stents.

FIG. 5 illustrates a catheter 34 placed into a chest wall 32 with medicated ink markings 14 made near the skin exit wound 33. The present invention enables a physician to apply the medicated ink marking 14 at a desired location on the medical device such as at or around the epidermal exit wound device contact area. For example, a user can apply antibiotic, analgesic, or anti-inflammatory medicated ink marks on a specific location of an indwelling catheter where the medicated ink marks will provide the most therapeutic benefit. Further, a user can also apply a medicated ink mark to the specific desired location of dialysis needles, dialysis catheters, orthopedic implant or traction pins, laparoscopic devices, or spinal tap needles with detectable confirmation and/or visual confirmation prior to or during medical device usage.

FIGS. 6A, 6B, and 6C illustrate ink application devices suitable for use with illustrative embodiments of the present invention. FIG. 6A illustrates one example embodiment of an ink jet printer 36. The ink jet printer 36 applies the medicated ink marking 14 to the medical device 12. An ink cartridge within the ink jet printer 36 can contain medicated ink for application by the ink jet printer 36. The dosage of medications, utilizing this method, can be digitally controlled in a predetermined pattern and shape of medicated ink mark made from the ink jet printer. In addition, different color ink cartridges can contain different types and classifications of medications based on different ink colors, as previously discussed. Further, the ink jet printer 36 can relatively accurately create simple color patterns using different colors, to provide additional identification for the particular medication or medications disposed within the ink.

FIG. 6B illustrates another embodiment in the form of a marker pen 38 containing a medicated ink. The marker pen 38 applies the medicated ink marking 14 to the medical device 12. Different color markers can contain different medication classifications or types of medication based on different color schemes. The marker pen 38 can also be utilized in forming simple color patterns.

FIG. 6C illustrates an ink pad device 40 embodiment. The ink pad device 40 applies the medicated ink marking 14 to the medical device 12. In addition, a different color ink pad can contain a different medication classification or type of medication based on different color schemes. Another application can utilize thermal transfer from a secondary film loaded with transferable medicated ink.

FIG. 7 illustrates an example method of determining an amount of medical agent to be applied to a medical device in accordance with an illustrative embodiment of the present invention. First, a user determines the amount of medical agent to be applied to a medical device (step 50). Second, the user determines dimensions of the visible marking to be applied to deliver the desired amount of medical agent (step 52). After applying a single medication mark (step 54), the user can apply different drugs to the same medical device as needed or apply more of the same medication with subsequent marker applications (step 56). The present invention can provide multiple medicated ink marks with different pharmaceutical effects and independent activation and/or release rates on a marked medical device.

FIGS. 8A, 8B, 8C, 8D, and 8E illustrate additional example embodiments of the medical device 12 that can make use of the teachings of the present invention. It should again be noted that the invention shall not be limited to these specific embodiments. These example structures are provided merely to illustrate the versatility of the medicated ink marking of the present invention.

FIG. 8A illustrates an example stent 60 as one form of the medical device 12. The stent 60 includes the medicated ink marking 14 along the side of the stent 60. In this instance, the medicated ink marking 14 provides an indication of the length and diameter of the stent 60, while also providing medication from the medicated ink marking 14 to a target location within a patient's body where the stent 60 is deployed.

FIG. 8B illustrates an example catheter 62 as another form of the medical device 12. The catheter 62 includes the medicated ink marking 14 at the end of the catheter 62. In this instance, the medicated ink marking 14 indicates a size of the catheter, either through a pattern or through color, and also provides medication to the puncture wound formed by the catheter 62.

FIG. 8C illustrates an example vascular graft 64. The medicated ink marking 14 resides on the side of the vascular graft 64 and indicates the length and diameter of the graft 64. The medicated ink marking 14 also provides a medicated agent to the patient's body along the surface of the vascular graft 64, as desired.

FIG. 8D illustrates an example surgical fabric or surgical mesh 66. The medicated ink marking 14 (in the form of a collection of circles having a predetermined color) provides information concerning the characteristics of the surgical mesh 66. The medicated ink marking 14 further provides a medicated agent to the patient's body at the location of the surgical mesh 66 placement. The medicated agent could be, for example, an agent that promotes tissue in-growth to anchor the surgical mesh 66 within the patient's body.

FIG. 8E shows another surgical mesh 68 of PET. Often, users of surgical mesh with a relatively larger section of mesh material must cut down that section to a smaller size to better fit the particular application. The user often utilizes a non-medicated ink marker and ruler to lay out a pattern for cutting the surgical mesh to size, shape, and orientation prior to and during use of the surgical mesh. In accordance with the present invention pre-printed lines 70 can be created on the mesh 68 to aid in cutting of the mesh 68, and reduce any errors in laying out the pattern to be cut. Further, the ink utilized in making the pre-printed lines 70 can be a medicated ink, if desired.

The medicated ink markings of the present invention enable the distribution of medication to a targeted location within a patient's body without adverse affect on the performance of the medical device upon which the ink is applied. The medicated ink is relatively thin and unobtrusive to the applied surface. The medicated ink can further provide relevant information concerning the medications contained within the ink and/or the medical device, as well as other characteristics of the ink and/or the medical device, such as drug type, drug brand, drug dosage, dimensions, sizing, placement, orientation, trimming, and the like. Because the medicated ink is placed directly on the medical device, misuse or mistaken identification of the medical device and its properties are substantially reduced because a user does not need to refer to removed packaging for identification information.

The present invention has many different therapeutic uses. More specifically, one clinical use for the medicated ink invention is for application onto implantable soft tissue medical devices for chest wall and abdominal wall repair. In particular, polypropylene mesh and porous surgical fabrics are placed in areas frequently subject to infection, inflammation, and organ tissue adhesion. Application of an identifiable and/or detectable medicated ink pattern on the surface of such polypropylene mesh and porous surgical fabrics provides a localized therapeutic solution for such complications following medical device implantation.

In particular, identifiable and/or detectable medicated ink containing anti-adhesion properties can be utilized for intraperitoneal surgeries where adhesion formation, or device attachment, to the bowel is undesirable. Application of an identifiable and/or detectable drug exuding ink containing anti-adhesion chemicals directly onto the polypropylene mesh provides desirable anti-adhesion properties at the tissue contacting site, maximizing the medication's effectiveness without systemic medication effects. A visible identification of the type, amount, and location in the form of a pattern can be provided with the medicated ink on the surgical mesh fabric. The clinical user (e.g., the surgeon) can then visibly orient the medical device with the medicated ink pattern specific to the clinical needs of the patient's anatomy and surgical installation.

Further, a surgeon may determine that more than one medication is required on the implantable device. Utilization of color differentiation for two distinctly different medications applied to the same medical device can be readily confirmed, or be used in the application of two different medicated inks onto one medical device. Use of color to distinguish two or more different medications with visual color coded medicated inks allows the physician to orient the medical device based on the needs of the patient's most therapeutic anatomical location. It should be noted that the identifiable and/or detectable medicated ink does not affect the porosity and/or biomechanical properties of the implantable medical device required for tissue ingrowth, tissue reinforcement, or reject encapsulation.

Application of the identifiable and/or detectable medicated ink onto polypropylene mesh, including hernia mesh plugs, urethral bladder neck suspension mesh or tape, thoracic chest wall meshes, lung volume reduction support material, aortic grafts, and abdominal wall tissue reinforcement implants, can include a variety of medications. The medications can improve infection resistance, minimize inflammation, limit adhesion of delicate organ tissues to the synthetic polymer mesh and/or influence foreign material cellular encapsulation. Antibiotic medications can include silver sulfadiazine, gentamycin, sirolimus, minocycline, paclitaxel, tacrolimus, everolimus vancomycin, ciprofloxacin, rifampin, mupirocin, RIP, kanamycin, hydroxyapatite, amikacin, ceftazidime, tobramycin, levofloxacin, bominated furonone, algae byproducts, doxorubicin, and chlorhexidine glyconate. The medications listed herein represent only a few examples of the type of medications that can be delivered locally by direct tissue contact with a medicated ink marking on a medical device. Other medications such as fibroblast growth factor and bone morphoneric protein can also be delivered by direct medical device contact that incorporates a medicated ink.

Different implantable medical devices can benefit from the use of medicated ink, for example, a vascular graft. Artificial arteries or synthetic vascular grafts typically are printed with a colored ink reference line that is used by the implanting surgeon for visual orientation and company identification. A visually detectable medicated ink printed as a reference line allows the surgeon to surgically orient the medical device so it is implanted in a straight and non-twisted condition. Such a drug exuding ink marking further provides a therapeutic benefit to the patient with the addition of numerous medications, i.e., antibiotic, anti-inflammatory, anti-proliferative, and agents of the like.

Application of the medicated ink can include drugs such as sirolimus, tacrolimus, everolimus, paclitaxel or vancomycin to control and/or limit cellular proliferation into and around the cell porous synthetic vascular graft. Use of such anti-proliferative antibiotics is also useful, as many vascular graft blunt dissection locations are frequently subject to topical bacterial contamination and chronic infection. Use of commonly prescribed antibiotics such as gentamycin, minocycline, or staphlococcal resistant antibiotics, such as kefzol and vancomycin, with the medicated ink helps prevent a vascular graft from becoming infected along its tissue tunnel following surgical implantation. Use of different colors, or another detection means to distinguish one medication and dose from another, allows the surgeon to confirm application, location, or type of medicated ink placed on the device. In addition, anatomical location indications for placement of the device at the time of implant can also be provided.

All such identifiable and/or detectable drug exuding inks can be made as a permanent marking or as a temporary marking, which can be absorbed by the local tissue.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. It is intended that the present invention be limited only to the extent required by the appended claims and the applicable rules of law.

What is claimed is:

1. A medical device comprising:
a structure adapted for insertion into a patient; and
a detectable marking applied to the structure, wherein the marking contains a medical agent for contacting body fluid when the device is placed within a patient.

2. The medical device of claim 1, wherein the marking is applied with a marker.

3. The medical device of claim 1, wherein a dosage of the medical agent on the device can be determined by detection of the marking.

4. The medical device of claim 1, wherein a dosage of the medical agent on the device is controlled by detectable dimensions of the marking on the device.

5. The medical device of claim 1, wherein a dosage of the medical agent on the device is determinable by visual detection of the marking.

6. The medical device of claim 1, wherein the device comprises an additional marking and wherein the marking comprises a first color and the additional marking comprises a second color that differs from the first color.

7. The medical device of claim 1, wherein the marking comprises more than one type of medical agent. 6

8. The medical device of claim 1, wherein the marking comprises at least one of a therapeutic and a diagnostic medical agent.

9. The medical device of claim 1, wherein the medical device comprises an implantable medical device.

10. The medical device of claim 1, wherein the medical device comprises an indwelling medical device.

11. The medical device of claim 1, wherein the medical device has a therapeutic function.

12. The medical device of claim 1, wherein the medical device has a diagnostic function.

13. The medical device of claim 1, wherein the medical device is placed into a patient's body for permanent use.

14. The medical device of claim 1, wherein the medical device is placed into a patient's body for temporary use.

15. The medical device of claim 1, wherein the medical agent comprises an antioxidant agent.

16. The medical device of claim 1, wherein the medical agent comprises an antihypertensive agent.

17. The medical device of claim 1, wherein the medical agent comprises an anti-inflammatory agent.

18. The medical device of claim 1, wherein the medical agent comprises a growth factor antagonist.

19. The medical device of claim 1, wherein the medical agent comprises an antiplatelet agent.

20. The medical device of claim 1, wherein the medical agent comprises an anticoagulant agent.

21. The medical device of claim 1, wherein the medical agent comprises a thrombolytic agent.

22. The medical device of claim 1, wherein the medical agent comprises drugs to alter lipid metabolism.

23. The medical device of claim 1, wherein the medical agent comprises an ACE inhibitor.

24. The medical device of claim 1, wherein the medical agent comprises at least one of an antiproliferative and an antineoplastic.

25. The medical device of claim 1, wherein the medical agent comprises a tissue growth stimulant.

26. The medical device of claim 1, wherein the medical agent comprises a chemical donor of at least one of Nitric oxide and Super Oxygenated O2.

27. The medical device of claim 1, wherein the medical agent comprises promotion of hollow organ occlusion or thrombosis agents.

28. The medical device of claim 1, wherein the medical agent comprises a functional protein and factor delivery agent.

29. The medical device of claim 1, wherein the medical agent comprises a second messenger targeting agent.

30. The medical device of claim 1, wherein the medical agent comprises an angiogenic agent.

31. The medical device of claim 1, wherein the medical agent comprises an anti-angiogenic agent.

32. The medical device of claim 1, wherein the medical agent comprises an inhibition of protein synthesis agent.

33. The medical device of claim 1, wherein the medical agent comprises an antiinfective agent.

34. The medical device of claim 1, wherein the medical agent comprises a gene delivery agent.

35. The medical device of claim 1, wherein the medical agent comprises at least one of a tissue perfusion enhancer and a tissue absorption enhancer.

36. The medical device of claim 1, wherein the medical agent comprises a nitric oxide donative derivative.

37. The medical device of claim 1, wherein the medical agent comprises drug carrying nano-particles.

38. The medical device of claim 1, wherein the medical agent comprises drug carrying micro-spheres.

39. The medical device of claim 1, wherein the medical agent comprises at least one of an imaging agent and a contrast agent.

40. The medical device of claim 1, wherein the medical agent comprises an anesthetic agent.

41. The medical device of claim 1, wherein the medical agent comprises a descaling agent.

42. The medical device of claim 1, wherein the medical agent comprises a cheomtherapeutic agent.

43. The medical device of claim 1, wherein the medical device comprises a stent.

44. The medical device of claim 1, wherein the medical device comprises a catheter.

45. The medical device of claim 1, wherein the medical device comprises a vascular graft.

46. The medical device of claim 1, wherein the medical device comprises a surgical mesh.

47. The medical device of claim 1, wherein the structure is adapted for implantation in the patient.

48. The medical device of claim 1, wherein the medical agent is suitable for release into the body of the patient.

49. The medical device of claim 1, wherein the medical agent is suitable for release into tissue of the patient.

50. The medical device of claim 1, wherein the medical agent includes an immobilized drug.

51. The medical device of claim 1, wherein the medical agent exudes from the marking.

52. The medical device of claim 1, wherein the medical agent is enclosed in liposomes.

53. The medical device of claim 1, wherein the ink marking is visible to the human eye without visual aid.

54. The medical device of claim 1, wherein the ink marking is visible to the human eye with use of a visual aid.

55. The medical device of claim 1, wherein the manner in which the ink marking is applied comprises at least one of a color code format, a shape format, a symbol format, and a size format to convey the information.

56. The medical device of claim 1, wherein the manner in which the ink marking is applied comprises application of the ink marking to indicate at least one of drug type, drug brand, drug dosage, dimensions, sizing, placement, orientation, and trimming guidelines.

53. A medical device comprising:
a structure adapted for insertion into a patient; and
a detectable information conveying marking applied to the structure, wherein the marking contains a medical agent for contacting body fluid when the device is placed within a patient.

54. A method of applying a detectable medicated information conveying marking to an implantable medical device, comprising the steps of:
providing an applicator holding detectable medicated ink; and
applying a first marking of the detectable medicated ink to the implantable medical device to apply a specific dosage of a drug, wherein the dosage is controlled by the quantity of the first marking.

55. The method of claim 54, further comprising pre-treating the medical device prior to applying the first marking.

56. The method of claim 54, further comprising applying a second marking detectably different from the first marking.

57. The method of claim 54, wherein applying the first marking comprises applying multiple drug medications to the implantable medical device.

58. The method of claim 54, wherein at least the first marking is applied to the implantable medical device by an ink jet printer.

59. The method of claim 54, wherein at least the first marking is applied to the implantable medical device by a marker pen.

60. The method of claim 54, wherein at least the first marking is applied to the implantable medical device by an ink pad device.

61. The method of claim 54, wherein at least the first marking is applied to the implantable medical device using thermal transfer.

62. The method of claim 54, wherein at least the first marking is applied to the implantable medical device using gas vapor deposition.

63. The method of claim 54, wherein the first marking comprises a first color.

64. The method of claim 63, further comprising a second marking formed of a second color that is visually differentiable from the first color.

65. The method of claim 54, wherein the medical device comprises a stent.

66. The method of claim 54, wherein the medical device comprises a catheter.

67. The method of claim 54, wherein the medical device comprises a vascular graft.

68. The method of claim 54, wherein the medical device comprises a surgical mesh.

69. The method of claim 54, wherein the medical ink is suitable for release into the body of the patient.

70. The method of claim 54, wherein the medical ink is suitable for release into tissue of the patient.

71. A method of applying a medical agent to a medical device, comprising the steps of:
determining a length of detectable information conveying marking to be applied to the device according to the amount of medical agent desired; and
applying the determined length of marking to the device.

72. The method of claim 71, further comprising pre-treating the medical device prior to applying the marking.

73. The method of claim 71, further comprising applying a different marking that is detectably different from the marking.

74. The method of claim 71, wherein applying the determined length of marking comprises applying multiple drug medications to the medical device.

75. The method of claim 71, wherein the marking is applied to the medical device by an ink jet printer.

76. The method of claim 71, wherein the marking is applied to the medical device by a marker pen.

77. The method of claim 71, wherein the marking is applied to the medical device by an ink pad device.

78. The method of claim 71, wherein the marking is applied to the medical device using thermal transfer.

79. The method of claim 71, wherein the marking is applied to the medical device using gas vapor deposition.

80. The method of claim 71, wherein the marking comprises a first color.

81. The method of claim 80, further comprising a different marking formed of a second color that is visually differentiable from the first color.

82. The method of claim 71, wherein the medical device comprises a stent.

83. The method of claim 71, wherein the medical device comprises a catheter.

84. The method of claim 71, wherein the medical device comprises a vascular graft.

85. The method of claim 71, wherein the medical device comprises a surgical mesh.

86. The method of claim 71, wherein the marking is suitable for release into a body of a patient.

87. The method of claim 71, wherein the medical ink is suitable for release into tissue of a patient.

88. A medicated stent system, comprising:
a stent structure adapted to be implanted in a patient; and
a detectable information conveying marking of ink applied to the stent structure, wherein the ink contains a medical agent to limit restenosis by release the medical agent when the stent is implanted.

89. The drug delivery stent system of claim 88, wherein the medical agent comprises at least one of Paclitaxel, Taxane, Sirolimus, Tacrolimus, Everolimus, and Mycophenolic acid.
